(19) United States
(12) Patent Application Publication
(10) Pub. No.: US 2010/0074965 A1
(43) Pub. Date: Mar. 25, 2010

(54) COSMETIC COMPOSITION COMPRISING A METEORITE EXTRACT, AND USE OF THE EXTRACT AS COSMETIC AGENT SUITABLE FOR STIMULATING THE DIFFERENTIATION OF HUMAN KERATINOCYTES

(75) Inventors: Murielle ERISSON nee CONRY, L'Isle D'Abeau (FR); Alain Guichon, Lyon (FR); Paula Lennon, Lyon (FR)

Correspondence Address: HESLIN ROTHENBERG FARLEY & MESITI PC, 5 COLUMBIA CIRCLE, ALBANY, NY, 12203, US

(73) Assignee: GATTEFOSSE SAS, Saint Priest (FR)

(21) Appl. No.: 12/418,854

(22) Filed: Apr. 6, 2009

(30) Foreign Application Priority Data
Sep. 22, 2008 (FR) 08 56344

(51) Int. Cl. A61K 8/19; A61Q 90/00

(52) U.S. Cl. 424/600

(57) ABSTRACT

The present invention relates to a cosmetic composition comprising a meteorite extract. The present invention also relates to methods of using the extract as a cosmetic agent suitable for stimulating the differentiation of human keratinocytes.

### CROSS REFERENCE TO RELATED APPLICATIONS

This application claims priority of French patent application No. 0856344, filed Sep. 22, 2008, the entire disclosure of which is hereby incorporated herein by reference.

The invention relates to a cosmetic composition comprising a meteorite extract. It also relates to the use of the extract as a cosmetic agent suitable for stimulating the differentiation of human keratinocytes.

### BACKGROUND

The review SÖFW Journal, 2003, 129(11), 18-22 reports extracts of stones issuing from terrestrial geological processes, in particular copper-rich malachite, zinc-rich smithsonite, and manganese-rich rhodochrosite. These extracts are used as cell protectors against oxidative stress caused by the environment (pollution, UV, tobacco, etc.). The action mechanisms of these 3 extracts are different, each of these 3 products acting at different cell levels:
the malachite extract protects the keratinocytes in the epidermis,
the rhodochrosite extract protects the fibroblasts in the dermis,
the smithsonite extract protects the DNA by stimulating metallothionein, a stress protein.

### SUMMARY OF THE INVENTION

The present invention relates to a cosmetic composition comprising a meteorite extract. The present invention also relates to methods of using the extract as a cosmetic agent suitable for stimulating the differentiation of human keratinocytes.

The Applicant has found that certain stone extracts available on the market did not allow the formulation of cosmetic compositions containing vegetable oils sensitive to oxidation.

The invention solves this problem by proposing a natural mineral extract not issuing from a terrestrial source but nevertheless containing the mineral elements which have demonstrated their effectiveness in the preceding extracts and are compatible with the formulation of vegetable oils sensitive to oxidation.

### DETAILED DESCRIPTION OF THE INVENTION

In the course of his researches, the Applicant has developed extracts not of stone but of meteorites, for which he has demonstrated the effect stimulating the differentiation of the human keratinocytes. It follows that the extract can be used as an anti-aging, epidermis restructuring, moisturizing or skin protection product.

According to the respective definitions of the Académie Française and Wikipedia, the free encyclopedia consulted on Mar. 6, 2008, stones and meteorites are clearly two distinct elements. Thus stone answers to the following definition of the Académie Française: “Hard and solid body employed in the construction of buildings, either detached from mountains or rocks, or extracted from the earth at a certain depth”, while Wikipedia defines it “in common language, as an element of the earth's crust, a pebble”. Meteorites, according to the Académie Française are “small bodies in movement in the earth's atmosphere, in intercosmic space. Aerolites are meteorites. Stony meteorites. Metallic meteorites. Powdery meteorites. Body moving in space and sometimes falling on the ground when it is not entirely consumed by friction with the atmosphere”.

Meteorites are essentially divided into two main categories, primitive meteorites and differentiated meteorites. Each of these categories has then been subdivided into groups (chondrites, achondrites and siderites) and each group into classes.

To the best of the Applicant's knowledge, not only has no meteorite extract been developed as of the date of the present invention, but also, and above all, meteorites have never been utilized in cosmetics.

In other words, the invention relates to a cosmetic composition comprising a meteorite extract, optionally in the presence of at least one cosmetically acceptable vehicle.

According to the invention, the extract originates advantageously from meteorites selected from the group comprising chondrites and achondrites. They are preferably and deliberately selected outside the extremely rare origins and exceptional compositions having a manifest importance for scientific investigations in astrophysics and for exceptional public collections.

In the group of chondrites, the meteorite is preferably selected from the class of ordinary chondrites and carbonaceous chondrites.

In the class of ordinary chondrites, this is advantageously the meteorite identified by the name NWA 869. The abbreviation NWA 869 corresponds to the 869^{th} meteorite officially recognized and referenced by the Meteoritical Society, among those found in northwest Africa. It has been the subject of a classification and a publication in the “Meteoritical Bulletin”, number 90 of September 2006 (Connolly, H. C. September 2006).

In the class of carbonaceous chondrites, this is advantageously the meteorite identified by the name Allende. The fall of this meteorite was observed on 8 Feb. 1969. After classification, it was given the name of the village Publico de Allende, in northern Mexico, where most of the fragments have fallen. It was published in the “Meteoritical Bulletin”, number 45 of February 1969 (Krinov, E. L. February 1969).

In the group of achondrites, the meteorite is preferably selected from differentiated achondrites, preferably from the group comprising the class of HED achondrites and that of asteroidal achondrites originating from other asteroids than Vesta.

In the class of Vesta achondrites, the meteorite identified by the name Millbillillie is particularly preferred. This meteorite was discovered on Millbillillie and Jundee, in Western Australia. Its fall was observed in October 1960 but its first samples were only found 10 years later, at coordinates 26°27′S, 120°22′E. It has been the subject of a publication in the “Meteoritical Bulletin” number 51 of June 1972 (Clarke, R. S. Jun. 30, 1972).

In the class of asteroidal achondrites, the meteorite identified by the name Al Haggounia is particularly preferred. This meteorite was identified in 2006 near the village of Al Haggounia in southern Morocco. It was the subject of a classification and publication in the “Meteoritical Bulletin” number 92 of September 2007 (Connolly, H. C. September 2007).

In practice, the meteorite is ground and the particles are then dissolved in a hot concentrated acidic or alkaline solution at a temperature between 40 and 100° C., advantageously a temperature between 60 and 85° C. The acids used are selected from the group comprising hydrochloric acid, citric acid, bromic acid, phosphoric acid or nitric acid, alone or in mixtures. The alkaline solutions used are selected from the group comprising potassium hydroxide and sodium hydroxide.

The next step consists in adding to the extract a sequestering agent or mixture of sequestering agents such as for example oxalate, sulfate, citrate, tartrate, salicylate, aspartate, gluconate or EDTA (ethylenediaminetetracetic acid).

The pH of the extract is then adjusted to the skin pH, in practice between 5.0 and 7.0, by adding a base such as potassium hydroxide, sodium hydroxide or sodium bicarbonate, or in case of alkaline attack, an acid such as hydrochloric acid.

The extract obtained is then filtered and stored in a sterile manner or by the addition of a preservative system, such as sorbic, benzoic, dehydroacetic acids and salts thereof, and also benzyl alcohol.

In an advantageous embodiment, the meteorite extract is in liquid form, the dry matter concentration preferably being between 10 and 15% by weight.

As previously indicated, the meteorite extract can be used as a cosmetic agent suitable for stimulating the differentiation of human keratinocytes, in particular as an anti-aging agent, epidermis restructuring agent, moisturizing agent, or skin protection agent.

In practice, the extract accounts for between 0.01 and 20%, preferably between 0.01 and 10%, advantageously between 0.1 and 5% by weight of the cosmetic composition.

The cosmetic composition of the invention is generally applied topically.

The cosmetic composition according to the invention may be in all galenic forms normally used for a topical application to the skin or hair, particularly in nearly anhydrous form, of an oil-in-water or water-in-oil or multiple emulsion, of a silicone emulsion or a microemulsion or nanoemulsion.

This composition may be more or less fluid and have the appearance inter alia of a white or colored crème, a pomade, a milk, lotion, a serum or a gel.

The composition of the invention may contain the usual additives in the cosmetic and dermatological fields, such as fats, emulsifiers and co-emulsifiers, hydrophilic or lipophilic gelling agents, hydrophilic or lipophilic active ingredients, preservatives, antioxidants, solvents, perfumes, fillers, hydrophilic and lipophilic filters, dyes, neutralizers, propenetrating agents, and polymers.

The quantities of these various additives are those conventionally used in the fields concerned, and for example 0.01 to 30% of the total weight of the composition. These additives, according to their type, may be introduced in the fatty phase or in the aqueous phase.

As fats usable in the invention, use can be made of mineral oils, oils of animal origin (lanoline), vegetable oils, synthetic oils (isopropyl myristate, octyldodecyl, isostearyl isostearate, decyl oleate, isopropyl palmitate), silicone oils (cyclomethicone, dimethicone) and fluorinated oils. Use can be made of fats such as fatty alcohols, fatty acids, waxes or gums and in particular silicone elastomers.

As emulsifiers and co-emulsifiers usable in the invention, mention can be made for example of esters of polyglycerols and fatty acid, esters of sucrose and fatty acid, esters of sorbitane and fatty acid, esters of fatty acid and oxyethylenated sorbitane, ethers of fatty alcohol and PEG, esters of glycerol and fatty acid, alkyl sulfates, alkyl ether sulfates, alkyl phosphates, alkyl polyglucosides, dimethicone copolyols.

As hydrophilic gelling agents, mention can be made in particular of carboxyvinyl polymers (carbomers), acrylic copolymers such as acrylate/alkylacrylate copolymers, polyacrylamides, polysaccharides such as xanthan gum, guar gum, natural gums such as gum of cellulose and derivatives, starches and derivatives thereof, clays and copolymers of 2-acrylamido-2-methylpropane acid.

As lipophilic gelling agents, mention can be made of modified clays such as bentones, metal salts of fatty acids, hydrophobic silica and ethyl cellulose.

The cosmetic composition may also contain active agents. As active agents, use can be made in particular of depigmenting agents, anti-free radical agents, emollients, moisturizers, anti-seborrheics, anti-inflammatories, anti-acne agents, keratolytic and/or desquamating agents, anti-wrinkle agents and tensors, draining agents, anti-irritants, soothing agents, thinning agents such as xanthic bases (caffeine), vitamins and mixtures thereof, matifying agents, anti-aging active agents such as retinol, cicatrizers, antiseptics and essential oils.

As preservatives usable according to the invention, mention can be made of benzoic acid, its salts and its esters; sorbic acid and its salts; parabens, their salts and esters; triclosan; imidazolidinyl urea; phenoxyethanol; DMDM hydantoin; diazolidinyl urea; chlorphenesin.

As antioxidants usable according to the invention, mention can be made of chelatants such as EDTA and salts thereof, sodium metabisulfite, sodium salicylate, sodium tartrate, sodium gluconate, trisodium citrate.

As solvents usable according to the invention, mention can be made of water, ethanol, glycerin, propylene glycol, butylene glycol, sorbitol.

As fillers usable according to the invention, mention can be made of talc, kaolin, mica, sericite, magnesium carbonate, aluminum silicate, magnesium silicate, organic powders such as nylon.

As filters usable according to the invention, mention can be made of UVA and UVB filters conventionally used such as benzophenone-3, butyl methoxydibenzoyl methane, octocrylene, octyl methoxycinnamate, 4-methylbenzylidene camphor, octyl salycylate, tacephthalydene dicamphor sulfanic acid, and drometrizole trisiloxane. Mention can also be made of physical filters TiO₂ and ZnO in their micron-sized and nanometer-sized forms, coated or uncoated.

As dyes usable according to the invention, mention can be made of lipophilic dyes, hydrophilic dyes, pigments and nacres commonly used in cosmetic or dermatological compositions, and mixtures thereof.

As neutralizing agents usable according to the invention, mention can be made of sodium hydroxide, triethanolamine, aminomethyl propanol, potassium hydroxide.

As propenetrating agents usable according to the invention, mention can be made of alcohols and glycols (ethanol, propylene glycol), ethoxydiglycol, alcohols and fatty acids (oleic acid), esters of fatty acids, dimethyl isosorbide.

The invention also relates to a method for the cosmetic treatment of skin aging, consisting locally applying an effective quantity of the cosmetic composition previously described, by topical application.

The invention and the advantages thereof will appear more clearly from the following examples.

### EXAMPLE 1

### Evaluation of a Meteorite Extract on the Differentiation of Cultured Normal Human Keratinocytes

### 1. Fabrication of a Liquid Extract of the Al Haggounia Meteorite

After grinding the meteorite, the particles are hot dissolved in a concentrated acid solution selected from strong acids. The solution is then sequestered. The pH of the extract is then adjusted to the skin pH by adding a base, the extract is then filtered. The final dry matter concentration of the extract is between 10 and 15% by weight.

### 2. Evaluation of the Biological Efficiency of the Product

The aim of this study is to evaluate the influence of the meteorite extract on keratinocyte differentiation in calcium-rich and calcium-free medium.

The study was carried out on normal human epidermal keratinocytes (NHEK) cultivated in a monolayer. The keratinocyte differentiation is estimated by measuring the expression of transglutaminase (TGK), an early marker of the differentiation. TGK, which catalyses inter-protein bonds during the formation of the corneal envelope, is a calcium dependent enzyme expressed by the keratinocytes cultivated in a monolayer.

General methodology. A preliminary cytotoxicity analysis is carried out by measuring the viability to MTT (3-(4,5-dimethylthiazol-2-yl)-2,5-diphenyl tetrazolium bromide) in order to determine the cytotoxicity threshold or maximum non-cytotoxic concentration of the products, after 96 hours of incubation, in the absence and presence of calcium. The total expression of TGK is evaluated on the control cultures and cultures treated for 96 hours, by measuring the fluorescence after marking the cells by an anti-TGK antibody and development by a secondary antibody coupled to a fluorochrome. The TGK content, expressed as fluorescence intensity per cell, is calculated after determining the number of cells in each culture well, by means of a second fluorescence measurement after counter-coloration of the nuclei by the Hoechst dye.

Results. Each condition is carried out in triplicate (n=3) for the test product and in hexaplicate (n=6) for the controls and references.

The intergroup comparisons are carried out using Student's test. The significativity of the results was evaluated at the 5% threshold (p<0.05). The statistic analysis can be interpreted if n>5. The data calculated for n≦5 are only indicative.

### A. Expression of TGK in Calcium-Free Medium—Non-Differentiating Conditions

| Concentration of | |
|---|---|
| meteorite extract | Expression of TGK |
| % v/v | % compared to control |
| 0.016% | 199% (s) |
| 0.00064% | 171% (s) |

0.016%: maximum non-cytotoxic concentration of meteorite extract
(s): p < 0.05

As shown by the table above, in calcium-free medium, the meteorite extract of the invention stimulates the expression of TGK compared to the control.

### B. Expression in TGK in Calcium-Rich Medium—Differentiating Conditions

| Concentration of | |
|---|---|
| meteorite extract | Expression of TGK |
| % v/v | % compared to control |
| 0.0064% | 134% (s) |
| 0.00128% | 144% (s) |
| 0.000256% | 147% (s) |

0.0064%: maximum non-cytotoxic concentration of meteorite extract
(s): p < 0.05

As shown by the table above, the meteorite extract of the invention stimulates the expression of TGK compared to the control, even when the control is rich in calcium, which is pro-differentiating.

In conclusion, in the experimental conditions of this study, the meteorite extract of the invention stimulates the expression of TGK, an early marker of keratinocyte differentiation, and therefore has a pro-differentiating effect.

### EXAMPLES 2

### Formulations

### Comparative Evaluation in Formulation of a Liquid Extract of Meteorite and a Liquid Extract of Malachite

The two formulas described below were formulated.

| Ingredients | Formula A | Formula B |
|---|---|---|
| PEG-6 Stearate, PEG-32 Stearate | 6.00 | 6.00 |
| Sweet almond oil | 15.00 | 15.00 |
| Stearic acid | 2.00 | 2.00 |
| Water | Qsp 100.00 | Qsp 100.00 |
| Carbomer | 0.20 | 0.20 |
| Sodium hydroxide | 0.04 | 0.04 |
| Meteorite extract according to | 0.014 | / |
| example 1 | (dry extract) | |
| Malachite extract | / | 0.014 |
| | | (dry extract) |

The 2 formulas were placed in closed glass bottles placed in an oven at 50° C. to simulate the aging of the product. After 5 days, the odors of the two formulas were evaluated.

### Result

Formula A containing a meteorite extract has an odor similar to the initial odor, whereas formula B has a strong odor of oxidized sweet almond oil. The meteorite extract can therefore be formulated in a formula containing unsaturated oils without giving rise to odor problems.

### Anti-Aging Crème

| Ingredients | Quantity (%) |
|---|---|
| Cetyl Alcohol, Glyceryl Stearate, PEG-75 Stearate, | 4.50 |
| Ceteth-20, Steareth-20 | |
| Cetostearyl alcohol | 1.00 |
| Cyclopentasiloxane | 3.00 |
| Octyldodecyl myristate | 2.00 |
| Borage oil | 1.00 |
| Octyl methoxycinnamate | 5.00 |
| Benzophenone-3 | 2.00 |
| Aluminum starch octenylsuccinate | 3.00 |
| Phenoxyethanol, Methylparaben, Butylparaben, | 1.00 |
| Ethylparaben, Propylparaben | |
| Carbomer | 0.15 |
| Xanthan gum | 0.20 |
| Disodium EDTA | 0.05 |
| Glycerin | 2.00 |
| Meteorite extract according to example 1 | 3.00 |
| Tocopherol acetate | 0.50 |
| Perfume | 0.30 |
| Water | Qsp 100 |

### Body Milk

| Ingredients | Quantity (%) |
|---|---|
| PEG-6 Stearate, Ceteth-20, Steareth-20 | 8.00 |
| Propylene Glycol Dipelargonate | 10.00 |
| Behenic acid | 1.00 |
| Jojoba oil | 2.00 |
| Sweet almond oil | 2.00 |
| Dimethicone | 1.50 |
| Cyclomethicone | 3.00 |
| Phenoxyethanol, Methylparaben, Butylparaben, | 1.00 |
| Ethylparaben, Propylparaben | |
| Acrylates/C10-30 Alkyl Acrylate Crosspolymer | 0.2 |
| Guar gum | 0.30 |
| Meteorite extract according to example 1 | 1.00 |
| Glycerin | 3.00 |
| Sodium hydroxide (10% solution) | 0.30 |
| Ascorbic acid | 0.05 |
| Perfume | 0.40 |
| Eau | Qsp 100 |

### O/W Emulsion

| Ingredients | Quantity (%) |
|---|---|
| Phenoxyethanol, Methylparaben, Butylparaben, | 1.0 |
| Ethylparaben, Propylparaben | |
| Magnesium aluminum silicate | 1.5 |
| Glycerin | 3.0 |
| Xanthan gum | 0.1 |
| Polysorbate-60 | 0.9 |
| Glyceryl Stearate, PEG-100 Stearate | 2.1 |
| Cetyl alcohol | 2.6 |
| Paraffin oil | 7.5 |
| Argan oil | 2.0 |
| Isopropyl myristate | 5.0 |
| Ethoxydiglycol | 5.0 |
| Meteorite extract according to example 1 | 5.0 |
| Perfume | 0.2 |
| Triethanolamine | 0.3 |
| Water | Qsp 100 |

### W/O Emulsion

| Ingredients | Quantity (%) |
|---|---|
| Glycerin | 3.0 |
| Propylene Glycol, Diazolidinyl Urea, Methylparaben, | 1.0 |
| Propylparaben | |
| Sodium chloride | 0.5 |
| Magnesium sulfate | 0.5 |
| Polyglyceryl-3 Diisostearate | 2.5 |
| Isohexadecane | 3.5 |
| Caprylic/Capric Triglyceride | 5.8 |
| Vaseline oil | 6.0 |
| Dimethicone | 4.0 |
| Perfume | 0.1 |
| Meteorite extract according to example 1 | 2.5 |
| Water | Qsp 100 |

### W/O/W Multiple Emulsion

| Ingredients | Quantity (%) |
|---|---|
| PEG-30 Dipolyhydroxystearate | 2.40 |
| Isohexadecane | 9.00 |
| PPG-15 Stearyl Ether | 4.50 |
| Caprylic/Capric Triglyceride | 4.50 |
| Magnesium sulfate | 0.82 |
| Propylene Glycol, Diazolidinyl Urea, Methylparaben, | 1.20 |
| Propylparaben | |
| Poloxamer 407 | 2.00 |
| Glycerin | 3.00 |
| Xanthan gum | 0.70 |
| Perfume | 0.20 |
| Malachite extract | 2.0 |
| Meteorite extract according to example 1 | 0.5 |
| Water | Qsp 100 |

### Sun Crème

| Ingredients | Quantity (%) |
|---|---|
| DEA Cetyl Phosphate | 2.0 |
| Glyceryl Stearate, PEG-100 Stearate | 4.0 |
| Octyl Methoxycinnamate | 6.0 |
| Butyl Methoxydibenzoylmethane | 2.0 |
| Benzophenone-3 | 1.0 |
| Titanium Dioxide | 2.0 |
| Butylenes glycol cocoate | 4.0 |
| Cyclomethicone | 3.0 |
| Tocopherol acetate | 0.5 |
| Disodium EDTA | 0.1 |
| Acrylates/C10-30 Alkyl Acrylates Crosspolymer | 0.2 |
| Xanthan gum | 0.3 |
| Phenoxyethanol, Methylparaben, Ethylparaben, | 1.0 |
| Propylparaben, Isobutylparaben | |
| Butylene Glycol | 3.0 |
| Sodium hydroxide (10% solution) | 0.4 |
| Perfume | 0.3 |
| Meteorite extract according to example 1 | 2.0 |
| Water | Qsp 100 |

### Foundation Crème

| Ingredients | Quantity (%) |
|---|---|
| Glyceryl Stearate, Propylene Glycol Stearate, Glyceryl | 5.00 |
| Isostearate, Propylene Glycol Isostearate, Oleth-25, | |
| Ceteth-25 | |
| Glyceryl Dibehenate, Tribehenin, Glyceryl Behenate | 1.00 |
| Hydrogenated castor oil | 1.00 |
| Ethoxydiglycol oleate | 6.00 |
| Isostearyl isostearate | 4.00 |
| Isopropyl myristate | 2.00 |
| Cetostearyl alcohol | 2.00 |
| Dimethicone | 5.00 |
| Tocopherol acetate | 0.50 |
| Phenoxyethanol, Methylparaben, Ethylparaben, | 0.60 |
| Propylparaben, Isobutylparaben | |
| Xanthan gum | 0.40 |
| Microcrystalline Cellulose, Cellulose Gum | 1.50 |
| Titanium Dioxide | 6.60 |
| Iron Oxides (Yellow pigment) | 1.55 |
| Iron Oxides (Red Pigment) | 0.43 |
| Iron Oxides (Black pigment) | 0.11 |
| Dimethicone, Dimethiconol | 3.00 |
| Rhodochrosite extract | 1.00 |
| Meteorite extract according to example 1 | 3.00 |
| Water | Qsp 100 |

### Serum

| Ingredients | Quantity (%) |
|---|---|
| Butylene Glycol | 4.00 |
| Glycerin | 2.00 |
| Meteorite extract according to example 1 | 0.50 |
| Acrylates/C10-30 Alkyl Acrylates Crosspolymer | 0.40 |
| Hydroxyethylcellulose | 0.20 |
| Disodium EDTA | 0.05 |
| Tocopherol acetate | 0.50 |
| Isostearyl alcohol, Butylene glycol cocoate, | 0.50 |
| ethylcellulose | |
| Phenoxyethanol, Methylparaben, Ethylparaben, | 0.70 |
| Propylparaben, Isobutylparaben | |
| Chlorphenesin | 0.20 |
| Sodium ascorbyl phosphate | 1.00 |
| Beech bud extract | 2.00 |
| Sodium hydroxide (10%) | 1.00 |
| Perfume | 0.20 |
| Water | Qsp 100 |

### Gel

| Ingredients | Quantity (%) |
|---|---|
| Propylene Glycol | 2.00 |
| Glycerin | 4.00 |
| DMDM Hydantoin, Iodopropinyl Butylcarbamate | 0.40 |
| Carbomer | 1.00 |
| Meteorite extract according to example 1 | 4.00 |
| Sodium hydroxide | 1.50 |
| Perfume | 0.15 |
| PEG-40 Hydrogenated castor oil, Polysorbate 20 | 0.50 |
| Water | Qsp 100 |

### Deodorant-Antiperspirant

| Ingredients | Quantity (%) |
|---|---|
| Hydroxyethylcellulose | 0.60 |
| Disodium EDTA | 0.10 |
| Aluminum hydrochloride | 10.00 |
| Ethoxydiglycol | 20.00 |
| Ethyl alcohol | 20.00 |
| Meteorite extract according to example 1 | 4.00 |
| Glycerin | 2.00 |
| PEG-40 Hydrogenated castor oil, Polysorbate 20 | 0.35 |
| Perfume | 0.15 |
| Water | Qsp 100 |

### Natural Emulsion

| Ingredients | Quantity (%) |
|---|---|
| Candelilla/Jojoba/Rice Bran Polyglyceryl-3 Esters, | 6.00 |
| Glyceryl Stearate, Cetearyl Alcohol, Sodium Stearoyl | |
| Lactylate | |
| Stearic acid | 2.00 |
| Squalane | 4.00 |
| Karite butter | 3.00 |
| Caprylic/Capric Triglycerides | 3.00 |
| Octyldodecanol | 4.00 |
| Jojoba oil | 3.00 |
| Vitamin E acetate | 0.50 |
| Glycerin | 5.00 |
| Microcrystalline Cellulose, Cellulose Gum | 2.50 |
| Xanthan gum | 0.30 |
| Sodium hydroxide (10% solution) | 1.40 |
| Dehydroacetic acid, benzyl alcohol | 0.80 |
| Fagus salvatica extract | 4.00 |
| Meteorite extract according to example 1 | 1.00 |
| Water | Qsp 100 |

### Lip Salve

| Ingredients | Quantity (%) |
|---|---|
| Caprylic/Capric Triglyceride | 72.2 |
| Jojoba oil | 15 |
| Glyceryl Behenate, Tribehenin, Glyceryl Behenate | 6 |
| Tocopherol | 0.1 |
| Hydrogenated Castor Oil | 1 |
| Glyceryl Stearate | 5 |
| Perfume | 0.2 |
| Meteorite extract according to example 1 | 0.5 |

### Toilet Water

| Ingredients | Quantity (%) |
|---|---|
| Water | Qsp 100 |
| Ethanol | 45 |
| Perfume | 5 |
| Meteorite extract according to example 1 | 0.5 |

### Eye Shadow

| Ingredients | Quantity (% w/w) |
|---|---|
| Glyceryl Dibehenate (and) Tribehenin (and) | 5.00 |
| Glyceryl Behenate | |
| Cetyl Alcohol | 6.00 |
| Caprylic/Capric Triglycerides | 41.00 |
| Titanium Dioxide | 3.00 |
| Talc | 24.00 |
| Ultramarines (and) Silica | 8.00 |
| Aluminum Starch Octenylsuccinate | 9.00 |
| Mica (and) CI 77491 (and) Triethoxycaprylylsilane | 4.00 |
| Meteorite extract according to example 1 | 0.5 |

### Lipstick

| | Quantity |
|---|---|
| Ingredients | (% w/w) |
| CI 15850 | 2.00 |
| CI 19140 | 1.70 |
| Titanium Dioxide | 1.65 |
| Butylene Glycol Cocoate | 6.00 |
| Ozokerite | 2.00 |
| Isostearyl Isostearate (and) Ozokerite (and) Castor Oil (and) | 78.65 |
| Hydrogenated Palm Kernel Glycerides (and) Propylene Glycol | |
| Dipelargonate (and) Polyglyceryl-2 Sesquiisostearate (and) | |
| PEG-8 Beeswax (and) Phenyl Trimethicone (and) Propylene | |
| Glycol Isostearate (and) Cetyl Lactate (and) Hydrogenated | |
| Palm Glycerides | |
| Hydrogenated Palm Kernel Glycerides (and) Hydrogenated | 4.00 |
| Palm Glycerides | |
| Polyglyceryl-3 Diisostearate | 2.00 |
| Meteorite extract according to example 1 | 2.00 |

1. A cosmetic composition comprising a meteorite extract.

2. The composition according to claim 1, wherein the meteorite is selected from the group consisting of chondrites and achondrites.

3. The composition according to claim 1, wherein the meteorite is identified by the reference NWA 869.

4. The composition according to claim 1, wherein the meteorite is identified by the name Millbillillie.

5. The composition according to claim 1, wherein the meteorite is identified by the name Allende.

6. The composition according to claim 1, wherein the meteorite is identified by the name Al Haggounia.

7. The composition according to claim 1, wherein the extract is liquid.

8. The composition according to claim 7, wherein the dry matter concentration is between 10 and 15% by weight.

9. The composition according to claim 1, wherein the extract accounts for between 0.0 1 and 20%, between 0.01 and 10%, or between 0.1 and 5% by weight of the cosmetic composition.

10. A method of using a meteorite extract as an anti-aging agent, skin restructuring agent, moisturizing agent or skin protection agent on a subject, said method comprising:
applying to a subject an effective quantity of the cosmetic composition according to claim 1 to stimulate differentiation of human keratinocytes of the subject.
